(19) United States
(12) Patent Application Publication
(10) Pub. No.: US 2026/0123821 A1
(43) Pub. Date: May 7, 2026

(54) ENDOSCOPIC DEVICE

(71) Applicant: Guy Aristide, East Meadow, NY (US)

(72) Inventor: Guy Aristide, East Meadow, NY (US)

(21) Appl. No.: 18/934,403

(22) Filed: Nov. 1, 2024

(51) Int. Cl. A61B 1/00; A61B 1/005; A61B 1/04; A61B 1/06; G16H 40/63

(52) U.S. Cl. CPC A61B 1/00108; A61B 1/000096; A61B 1/00016; A61B 1/00042; A61B 1/00066; A61B 1/00103; A61B 1/00105; A61B 1/00119; A61B 1/00131; A61B 1/00147; A61B 1/0055; A61B 1/04; A61B 1/0669; G16H 40/63

(57) ABSTRACT

Apparatus and methods for an endoscopic patient safety device that uses video monitoring and machine learning technology to identify abnormalities of a patient's respiratory system and provides automatic insertion, placement, and navigation of the endoscopic patient safety device into the patient's body are described. The endoscopic patient safety device automatically determines a patient's anatomy and retracts and expands to place its tip in an optimal zone for performing aspiration and other medical tests. Enroute to being inserted without having to be held, the endoscopic patient safety device uses sensors and machine learning to regulate its insertion speed, pauses, and expansion and retraction based on abnormalities encountered along the way. The data collected by the device is reported wirelessly for further analysis.

### FIELD OF INVENTION

Embodiments of the present disclosure relate to an endoscopic patient safety device generally, and more particularly to, using video monitoring and machine learning technology to identify abnormalities of a patient's respiratory system and to control the endoscopic patient safety device, also referred to as a wireless endoscope, while collecting data. Some embodiments or aspects may relate to other features, functionalities, or fields.

### BACKGROUND

In general, an endoscopic device is used to look inside the human anatomy by way of a body cavity. A portion of the device is inserted through a body cavity, such as the nasal cavity or mouth, to take pictures or videos of organs and other structures. Clinicians use endoscopic devices to screen, diagnose, and treat conditions.

One such use of an endoscopic device is to screen, diagnose, or treat aspiration or near aspiration events. Multiple different evaluations are employed by clinicians in order to evaluate aspiration events. One such examination is the modified swallowing test. The modified swallowing test permits clinicians to observe anatomical structures in the mouth and throat, as they are actively functioning when a patient is chewing, drinking, and swallowing.

Another examination employed is the Flexible Endoscopic Evaluation of Swallowing with Sensory Testing (FEES) which is a technique used to directly examine motor and sensory functions of swallowing so that proper treatment can be given to patients with swallowing difficulties to decrease their risk of aspiration and choking. During this examination, a clinician passes an endoscopic device through a patient's nose while the patient swallows liquids and foods of varying consistencies to assess the patients swallowing function.

Existing endoscopic devices can be bulky and difficult to use. Current administration of an endoscopic device requires a care provider to be bed side, holding the device the entire time it takes to administer the required tests. The existing endoscopic devices also require the device to be connected to a display in order for the physicians to accurately place the device within the patient.

The existing endoscopic devices also require the physician to be bedside and hold the device during the entire administration of the exam. Such constraints result in locking up the physician's time for a single patient thereby adding needless expenditure of hospital resources.

Typically, when a physician is placing the device within a patient, the device is placed based on the physician's experience and judgment. Relying on the physician's experience and judgment oftentimes leads to missed diagnosis. The physician may be limiting the exam to the incorrect part of the anatomy, missing areas of concern. Further, the physician may be under a time constraint and may only have time to limit the test to one area of the anatomy causing a missed diagnosis. Even further, due to the physician concentrating on placing the device within the correct area of the anatomy of the patient, the physician may overlook areas of concern due to the physician having to concentrate on multiple tasks.

Constantly having to hold the device during administration of an exam, using judgment in an attempt to properly place the device within the patient, and the device having to be directly connected to a display allows for many opportunities for errors and for harm to occur.

Increasing the resources available to physicians, such as minimizing multi-tasking while administering the examinations, and increasing the amount of time of the examination, can be improved to reduce the considerable clinical and economic burden of missed diagnosis.

Accordingly, there is a need for an endoscope that provides additional features and flexibility of use to address some of the above-mentioned drawbacks.

### BRIEF DESCRIPTION OF THE DRAWINGS

The various objects and advantages of the disclosure will be apparent upon consideration of the following detailed description, taken in conjunction with the accompanying drawings, in which like reference characters refer to like parts throughout, and in which:

FIG. 1 illustrates an overall system view of an aspiration device placed in a patient while laying on a hospital bed in which the device is sending video to a display device, in accordance with some embodiments of the disclosure;

FIG. 2 is a block diagram of an example system for using the endoscopic device, in accordance with some embodiments of the disclosure;

FIG. 3 illustrates an exploded view of the endoscopic device, in accordance with some embodiments of the disclosure;

FIG. 4 illustrates an exploded view of the endoscopic device with a motor included, in accordance with some embodiments of the disclosure;

FIG. 5 illustrates a proximal perspective view of the endoscopic device, in accordance with some embodiments of the disclosure;

FIG. 6 illustrates a distal perspective view of the endoscopic device, in accordance with some embodiments of the disclosure;

FIG. 7 is an example data from multiple patients, in accordance with some embodiments of the disclosure;

FIG. 8 is a flowchart of a process for analyzing patient data to detect abnormalities, in accordance with some embodiments of the disclosure;

FIG. 9 is a flowchart of a process for determining position and orientation of the distal tip of the endoscopic and auto-retraction and auto-expansion to make any adjustments, in accordance with some embodiments of the disclosure;

FIG. 10 is a flowchart of a process for determining position and orientation adjustments of the endoscopic device, in accordance with some embodiments of the disclosure;

FIG. 11 is an illustration depicting the optimal zone within a patients anatomy of where the distal tip of the endoscopic device should be placed, in accordance with some embodiments of the disclosure; and

FIG. 12 is an illustration of an exemplary user interface displaying an identified abnormality within the patient's anatomy.

### DETAILED DESCRIPTION

Hereinafter, the present invention will be described with reference to the accompanying drawings. The present invention may have various embodiments, and modifications and changes may be made therein. Therefore, the present invention will be described in detail with reference to particular embodiments shown in the accompanying drawings. However, it should be understood that there is no intent to limit the present invention to the particular forms, and the present invention should be construed to cover all modifications, equivalents, and/or alternatives falling within the spirit and scope of the present invention.

In accordance with some embodiments disclosed herein, some of the above-mentioned limitations are overcome by providing a wireless endoscopic device, also referred to as a wireless endoscope, that may be used to screen, diagnose, or treat aspiration, conduct swallowing tests, or scan for polyps and other protrusions in the patient's mouth and throat, and observe anatomical structures in the mouth and throat, including detect cancerous structures, without requiring constant holding of the endoscopic device, contact monitoring and adjusting insertion and depth of insertion of the endoscopic device, and relying solely on human skill that may vary from caregiver to caregiver. Some of the above-mentioned limitations are also overcome by providing a wireless endoscopic device that automatically orients, extends, and adjusts based on a patient's anatomy to ensure that the endoscopic device is inserted in the right areas within the patient's mouth and throat to accurately conduct the tests and screenings described above.

The endoscopic device, in some embodiments, may be a reusable, wireless, and machine learning technology enhanced endoscopic patient safety device that allows physicians and other integral care providers to review collected data remotely without disrupting the flow of their practice.

The endoscopic device is a wireless device. It is not directly connected via physical wire while in use. It may be placed within the patient's body cavity and left to monitor the patient allowing the care provider to focus on high-risk alarms during assessment flagged by the machine learning technology that recognizes aspiration or near aspiration events. A Bluetooth enabled camera may be located near the distal insertion end of the device, allowing for the collected video data to be sent from the device to a display in real time. The physician is not required to be bedside during the entire administration of the examinations. Rather, for example, the physician may place the device within the patient's body cavity, leave the room, and allow for the device to collect and send the data of the patient's anatomy to a display device. The device may also automatically search for and wirelessly connect to other authorized devices for transmitting the collected data to a display of the connected device.

Further, allowing for the unattended collection of data of the patient's anatomy allows for more data to be collected over a longer period of time. It also allows physicians to focus on other tasks, such as reviewing the data, such as video, collected by the device, allowing the physician's sole focus to be understanding the patient's anatomy and identifying abnormalities.

In some embodiments, the device may determine the area within the esophagus in which the device may monitor. The device may auto-retract or auto-expand based on the determined optimal zone in which the device is to monitor. In this manner, the device makes a determination without human intervention on the area within the patient's anatomy of where the monitoring should take place.

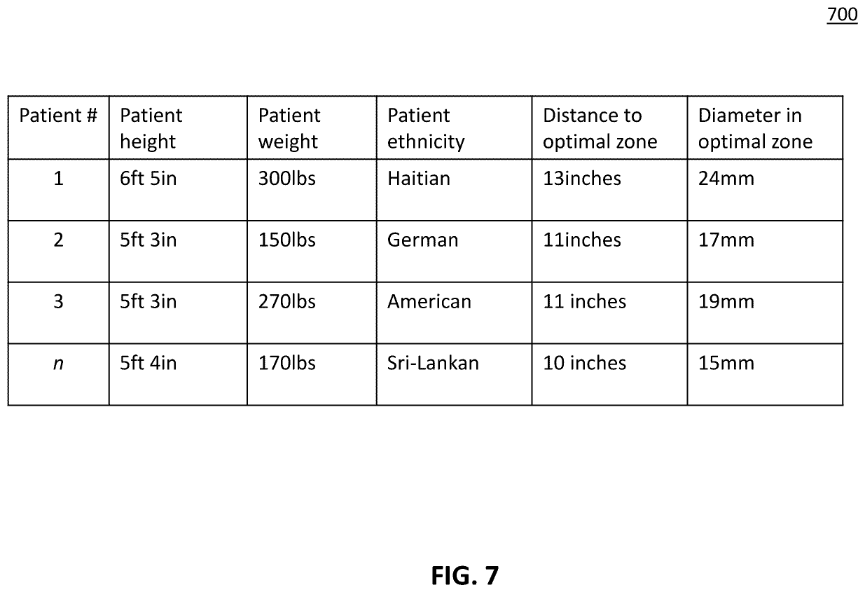

Typically, when administering examinations, physicians place the device in a specified area within the patient's anatomy. This specified area varies from person to person based on the patient's size. The device may be able to recognize the best position for the device to reside based on the machine learning technology. Since each patient's anatomy differs, e.g., a 6 ft 7 in person's throat size may be different than a 3 ft 5 in person or from a child, the device is capable of automatically collecting data while being inserted into a patient and automatically adjusting its length, orientation, angles and other type of positioning to accommodate for the patient's anatomy. An example of differing patient throat sizes and their body measurements is described in FIG. 7. To do so, the endoscopic device may leverage artificial intelligence (AI) engines executing AI platforms to determine appropriate positioning and orientation for the patient and accordingly adjust itself automatically. In some embodiments, the device may be in different sizes. For example, a smaller size may be used for children and a larger size may be used on adults. The size of the device may also vary based on the anatomy of the person, for example, the device size for a 7 ft adult may be different than a 5 ft adult.

Once the device is placed, the device tip may also orient automatically, by adjusting the angle of the tip of the device, to ensure that the device is collecting the correct data. The lights of the device may also auto-adjust the brightness to ensure the visibility of the patient's anatomy. The lights of the device may be placed at any portion of the device that allows for the light to shine inside the patient's body cavity.

In some embodiments, the lights of the device may also be used to screen for oral cancers leading to early detection. There may be several methods to use the lights for detecting cancers. For example, the lights may illuminate inside the patient's body cavity to allow a physical to clearly examine any abnormalities. The illumination may also allow the system to automatically capture images based on abnormalities that are detected based on better visibility due to the lights. In other embodiments, lights may use specific wavelengths to excite certain molecules in tissues to detect cancers. Since cancerous tissues may exhibit different fluorescence properties compared to healthy tissues, by emitting light at certain wavelengths the physician or the system may be able to identify potential cancers. Other lighting techniques that can contrast between healthy and abnormal tissue may also be used to detect cancers. For example, a technique that uses a blue light device that uses Fluorescence Visualization (FV) to detect oral cancer may be used. Using FV, healthy tissue may appear light green, while abnormal tissue appears dark. In terms of use, light from the device may be an be placed into the mouth by an ENT doctor to help detect lesions, white and red patches, and problem areas that are not visible under white light.

While monitoring the patient's anatomy, the device, using machine learning technology, collects data, such as video and pictures, and determines if any abnormalities are detected. If an abnormality is detected, the physician may be notified of such abnormality.

The system may also identify the abnormality, for example, by outlining the detected abnormality on the display screen. Using machine learning technology to analyze such collected data, allows for decreased chance of error of missed diagnosis and also increases hospital resources and expenditure.

As used herein, the term “physician” or “caregiver” refers to any personnel that may be responsible for using the device. For example, the terms “physician” or “caregiver” as used herein, may be interchangeable with, for example, the terms doctor, medical practitioner, surgeon, nurse, custodian, attendant, resident, or any other person who may be responsible for using the device. The terms are only meant to be used for exemplary purposes.

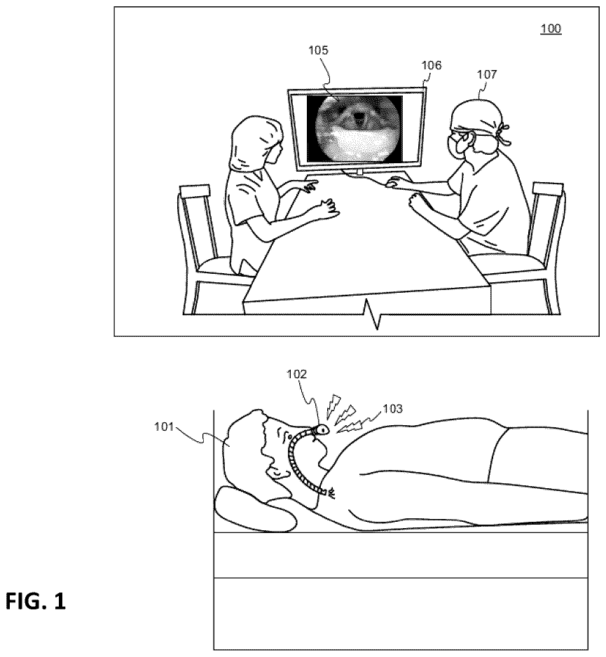

Referring not to figures, FIG. 1 illustrates an overall system 100 of the endoscopic device 103 inserted into a patient's body cavity 101 sending video data 105 via a wireless connection, such as Bluetooth or Wi-Fi connection 104, to a display device 106. In this example, the device 106 is placed in the patient's 101 nasal cavity in order for the patient's anatomy to be examined. The device 103 is left unattended while the device 103 collects data. The collected data 105, such as video, pictures, etc., are sent to a display device 106 to be analyzed by machine learning technology. In one embodiment, the physician 107, may be in a separate room from the patient 101 while device 103 is collecting data. Since the device may transmit the data wirelessly to another device or display, which may be located in the physician's vicinity, the physician 107 may be able to access the display or another device and focus on analyzing the video 105 in real time as the data is sent from the device 103 to the display 106.

In one embodiment, the device 103 is adhered to the patients 101 nose with an adhesive to ensure that the device 103 is not moved while collecting data. The device 103 may be adhered to the patient 101 in any suitable manner to ensure that the device 103 does not become disconnected from the patient.

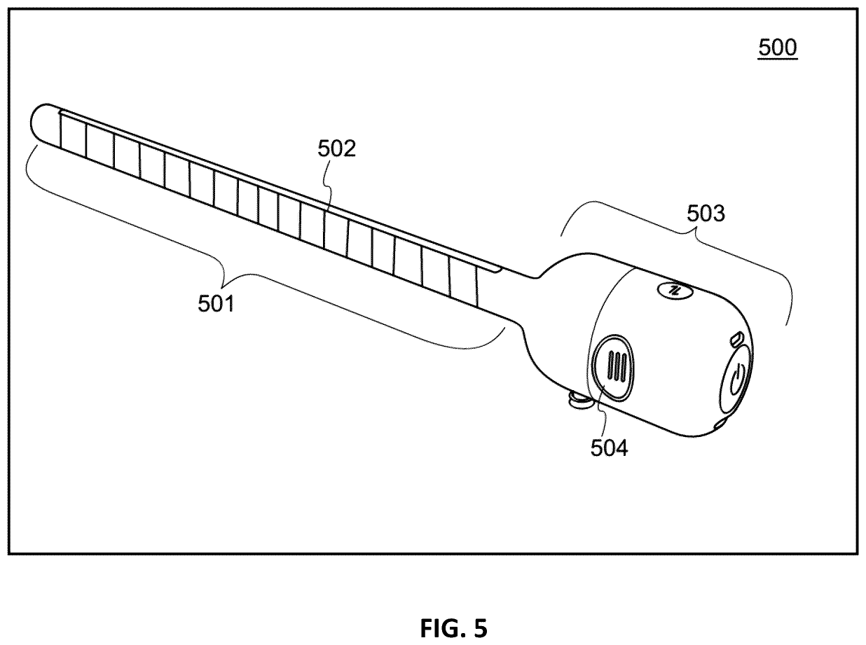

In one embodiment, once the device 103 is inserted into a patient's 101 body cavity, and the device 103 is bent to a certain angle, the curvature of the device will ensure that the device 103 does not become displaced while in the patient 101. If a physician begins to physically remove the device 103 from the patient, the device 103, through a sensor, will detect a threshold amount of pressure that is being applied to the device 103 and the device will return to its straight position as shown in FIG. 5 for ease of removal. In another embodiment, the device may include a release button that may be touched or pressed which causes the device to automatically retract and fall off or be removed from the patient. The device 103 may also include a clip or other attachment means to the patient such that it does not fall off once inserted. In some embodiments, the device 103 may include a gyroscope, either in the first or second portion, which may be used to prevent the device from falling off. For example, when the device 103 tilts, the gyroscope may sense the tilt and stabilizers in the device 103. The endoscopic device may obtain readings from the gyroscope and activate stabilizers, such as motorized stabilizers, balancers, to automatically readjust weights within the device 103 to shift the device's center of mass and ensure it does not fall off from the patient's nose. For example, a weight positions on a slider inside the tubular portion may be moved to its distal end, e.g., the distal end being inserted such as into the throat of the patient, such that it is weighted heavier preventing based on the force of gravity for the device to slide out or fall out from the patient. The device may also include an adhesive attachment to attach it to the patients after its inserted in the nose.

In another exemplary embodiment, the device will send the collected data 105, via, for example, Bluetooth 104, from the device 103 to the display device 106. Once the collected data 105 is received, machine learning technology will analyze the video 105 in order to detect any abnormalities that may be present in the patients 101 anatomy.

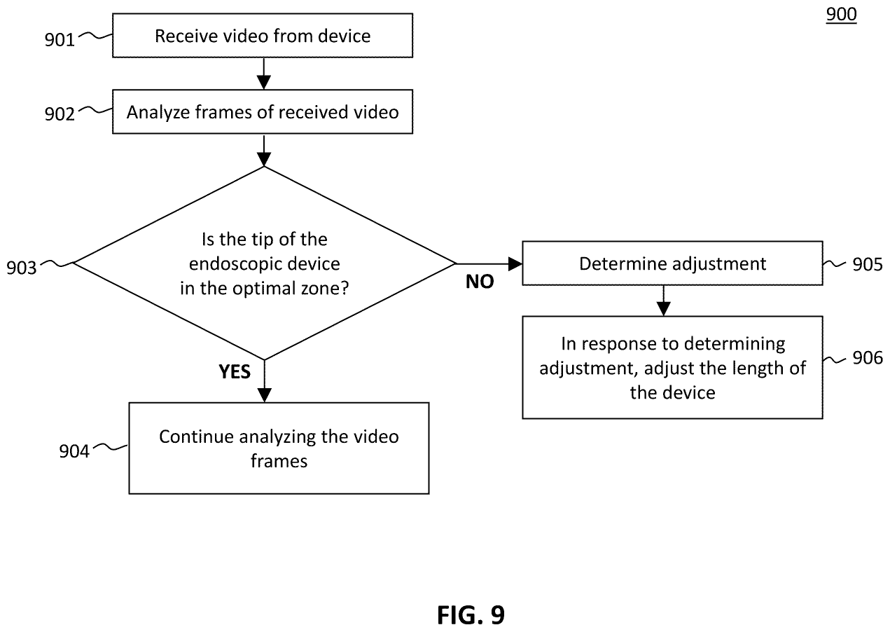

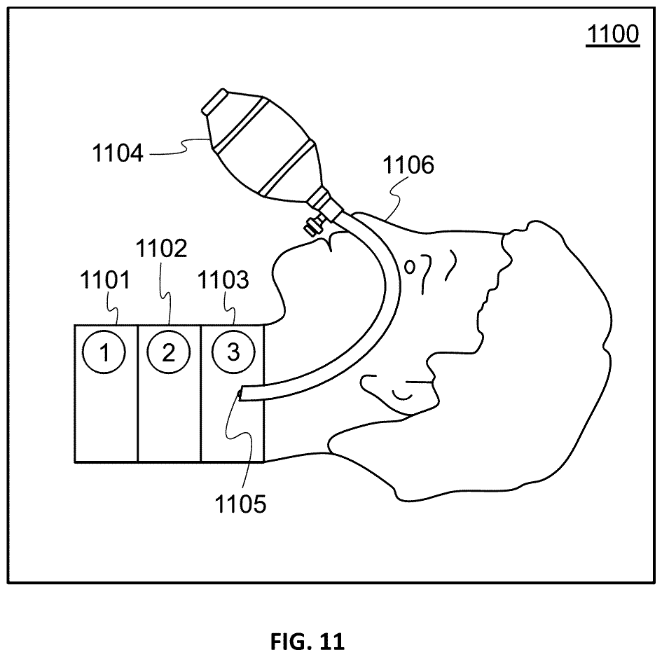

In one embodiment, the machine learning technology will analyze the collected data 105, such as video data sent from the device 103, to determine if the device 103 is placed within the optimal zone as illustrated and further described in FIG. 9 and FIG. 11. Using machine learning technology, if the device 103 is not placed within the optimal zone, the device 103 will be sent a command signal, in which the device 103 may either expand or retract the length of the device automatically without any human intervention. This enables the physician 107 to continue to analyze the video data 105 without any multi-tasking or interruptions.

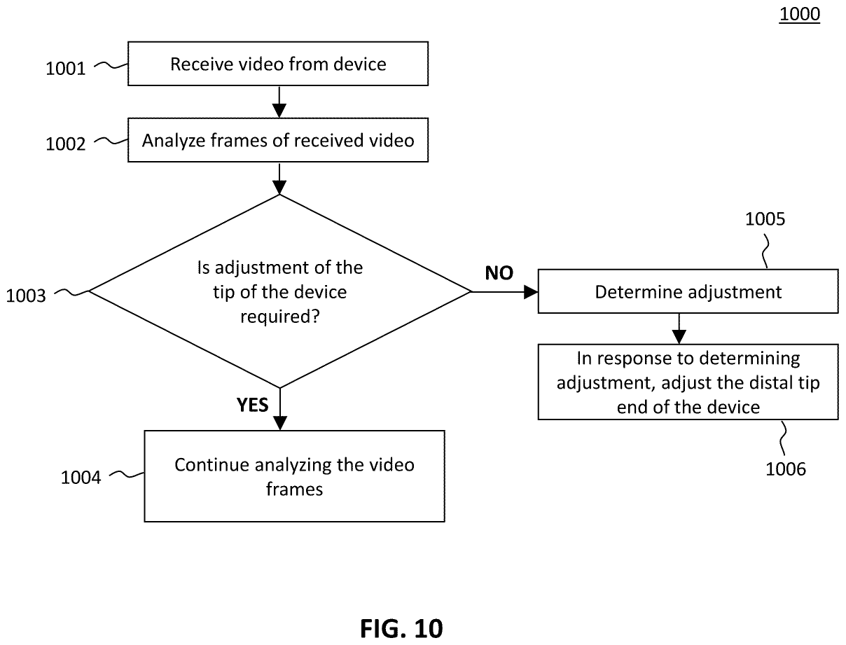

In one embodiment, the machine learning technology will analyze the collected data 105, such as video data sent from the device 103, to determine if the angle of the tip of the device 103 should be adjusted as illustrated and further described in FIG. 10. Using machine learning technology, if the tip of the device 103 should be adjusted to a different angle, the device 103 will be sent a command signal, in which the device may adjust the tip to the determined angle. This tip adjustment is performed without any human intervention, enabling the physician 107 to continue to analyze the video data 105 without any multi-tasking or interruptions.

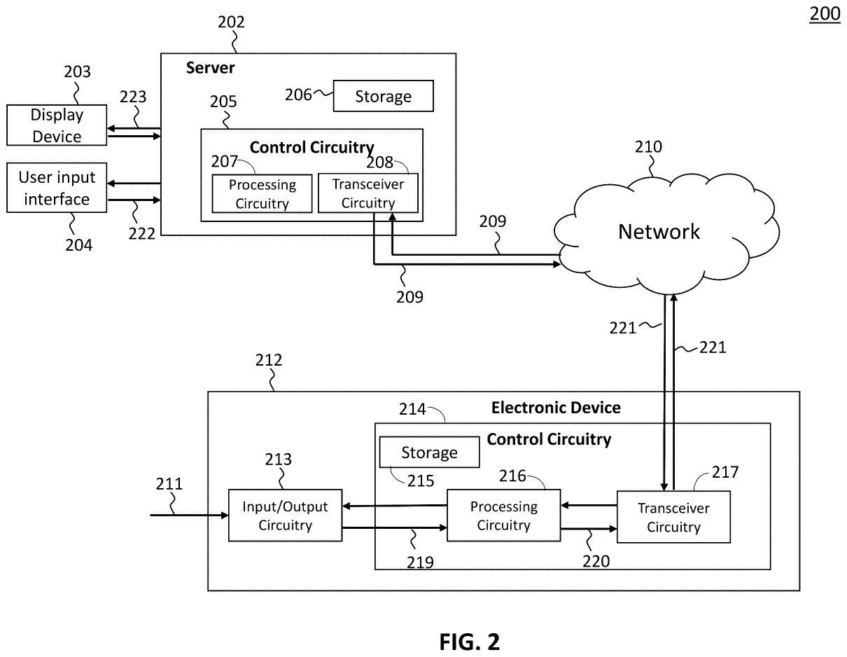

Referring now to FIG. 2, system 200 is a simplified illustrative overall system for using an endoscopic device to collect data and to send the collected data to a user interface display. In one embodiment, the machine learning technology may analyze the collected data and send command signals to the device and/or provide notification signals of identified abnormalities of the patient's anatomy. This exemplary system comprises electronic device 212, server, 202, display 203, and user input interface 204.

Electronic device 212 may receive control commands and send data via input/output (hereinafter “I/O”) path 213. I/O path 213 may provide control commands (e.g., commands to expand the length of the device, retract the length of the device, adjust the angle of the device, take pictures, adjust the brightness of the light source, and other commands) to control circuitry 214, which includes processing circuitry 216, transceiver circuitry 217, and storage 215. Control circuitry 214 may be used to send and receive commands, requests, and other suitable data using I/O path 213. I/O path 213 may connect control circuitry 214 (and specifically processing circuitry 216 and transceiver circuitry 217) to one or more communications paths 221. I/O functions may be provided by one or more of these communications paths 221 but are shown as a single path in FIG. 2 to avoid overcomplicating the drawing.

Control circuitry 214 may be based on any suitable processing circuitries such as one or more microprocessors, microcontrollers, digital signal processors, programmable logic devices, etc. In some embodiments, control circuitry 214 executes instructions for an electronic device stored in memory (i.e. storage 215).

In system 200, the electronic device may be coupled to network 210. Namely, electronic devices and servers are coupled to network 210 via communications paths. Network 210 may be implemented by any medium or mechanism that provides for exchange of data between devices in the communication system 200. Examples of networks include without limitation, a network such as a Local Area Network (LAN), Wide Area network (WAN), the internet, one or more terrestrial, satellite, or wireless links, etc. Alternatively, or additionally, any number of devices connected to network 210 may also be directly connected to each other through a communications link such as short range point-to-point communication paths, IEEE 1394 cables, wireless paths (e.g., Bluetooth, infrared, IEE 802-11x, etc.), or other short-range communication via wired or wireless paths. BLUETOOTH is a trademark owned by Bluetooth SIG, INC. The electric device 212 may also communicate with the server 202 directly through an indirect path via network 210.

A user may control the control circuitry 214 using user input interface 204. User input interface may be any suitable user interface such as mouse, trackball, keypad, keyboard, touch screen, touch pad, stylus input, joystick, voice recognition interface, or other user input interfaces. Display 203 may be provided as a stand-alone device or integrated with other elements of user input interface 204. Display 203 may be one or more of a monitor, a television, a liquid crystal display (LCD) for a mobile device, or any other suitable equipment for displaying visual videos and images.

System 200 is intended to illustrate network configuration by which electronic device 212 and server 202 may communicate with each other for the purpose of sending data and commands. Server 202 may include one or more processing circuitry 207, transceiver circuitry 208, and storage 206. Sever 202 may be any suitable combination of hardware and software capable of interactions with electronic device 212, display device 203, and user input interface 204. Server 202 may, for example, receive data from electronic device 212 for processing. Processing circuitry 207 may include any suitable processor, such as a microprocessor or group of microprocessors, and other processing circuitry such as caching circuitry, direct memory access (DMA) circuitry, and input/output (I/O) circuitry.

Storage 206 may include any suitable storage device including memory or other storage devices, such as random access memory (RAM), read only memory (ROM), flash memory, and a hard disk drive, that is suitable for storing data.

Server 202 may connect control circuitry 205 (and specifically processing circuitry 207 and transceiver circuitry 208) to one or more communications paths 209. Server 202 functions may be provided by one or more of these communications paths 209 but are shown as a single path in FIG. 2 to avoid overcomplicating the drawing.

The present invention may be applied in any one or a subset of these approaches, or in a system employing other approaches for delivering data and commands between the server 202 and the electronic device 212.

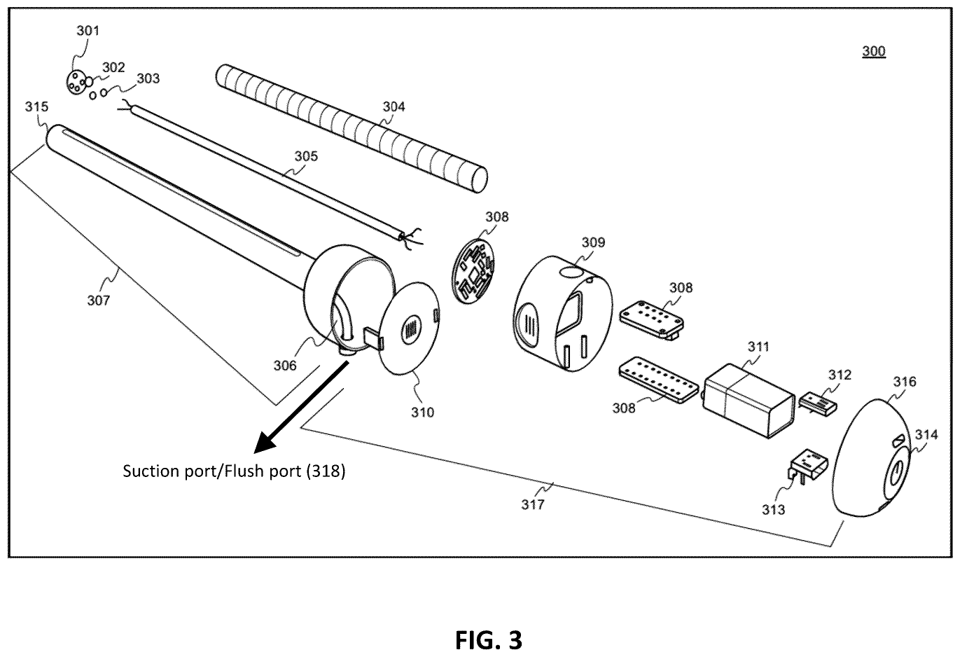

FIG. 3 is an exploded view of the endoscopic device 300. The endoscopic device 300 may comprise two sections, for example, a first section 307, and a second section, 317. The first section 307 and the second section 317 may be connected, for example, by a snap lock 309, or any other suitable lock that joins the two sections together, e.g. twist-on connection, fastener connection, latch connection etc. In one embodiment, the first section 307 is inserted into the patient's body cavity and is placed within the patient's anatomy in order for device 300 to collect the data. The first section may include a tip 301, one or more cameras 302, one or more light sources 303, electrical wires 305 connecting the one or more cameras 302 and one or more light sources 303 to the electrical connector 310, rings for bending of the device 304, and one or more suction channels 306.

In one embodiment, the first section 307 may be disposable. For example, after using the device 300, the first section 307 may be removed via the snap lock 309 from the second section 317. The first section 307 may be disposed of after its initial use. In another embodiment, the first section 307 is reusable.

In one embodiment, the first section 307 may bend, expand, and retract based on the commands received from the control circuitry 205. For example, while the first section 307 of the device 300 is inserted into the patient's anatomy and the camera 302 is collecting data, the processor 216 may process a command received from the server 202, that the distal end 315 of the first section 307 of the device 300 is not located in the optimal position within the patient's anatomy. The command may instruct the device to retract a certain amount to a specified location to be in the optimal zone.

In one embodiment, the second section 317 is located outside of the patient's anatomy while the device 300 is in use. The second section 317 may include electrical connector 310, at least one PCB 308, a connection piece 309 such as a snap lock, a battery 311, USC Type-C connection 312, HDMI connection 313, and a power button 314. The second section 317 may be reusable. In another exemplary embodiment, the second section 317 is disposable.

In one embodiment, the first section 307 of the endoscopic device 300 at the distal end 315 is the tip 301, the camera, 302, and the light source 303. The tip 301 may be a domed tip or any suitable shape necessary for ease of insertion into the patient body cavity.

In one embodiment, positioned within the tip 301 may be the camera 302 and the lighting source 303. The camera 302 may be used to collect data such as video or picture data of the patient's anatomy while the device 300 is placed within the patient's body cavity. The camera 302 may be Bluetooth enabled in order to send the collected data to a display device. The camera 302 may also include anti-fog technology to ensure that the imagery is visible at all times. The device 300 may not be directly connected while in use and may send the collected data over Bluetooth or Wi-Fi connection. In another exemplary embodiment, one or more cameras may be included within the device 300. For example, located within the distal end 315 of the tip 301 two cameras 302 could be included in order to provide for more collection of data. The lighting source 303 may be any suitable light source necessary to provide visibility while the device is inserted into the patient's body cavity, for example, the light source may be LEDs. In another embodiment, the light source may adjust the brightness of the lights automatically in order for the video that is being collected to remain visible. In another embodiment, the physician may make manual adjustments to the brightness of the light source. The light source may also be any suitable color necessary in order to diagnose, prevent, monitor, examine, and/or treat a disease or injury.

In one embodiment, also located within the first section 307 of the endoscopic device 300 are rings for bending of the device 304. A suction channel 306 may also be located within the first section 307 of the device 300. Electrical wires connected to the camera 302 and lighting source 303 are connected to the electrical connector 310 located in the second section 317 of the device 300. In some embodiments, a suction port or flush port 318 may be attached to the suction channel 306. In some embodiments, the flush port may be used to deliver water, saline, or other liquid solutions to aid the visualization. Once inserted, the solution may clear any debris, mucus, or other obstructions to allow for a clear examination. The same may be performed using a suction port. In other embodiments, the suction port may also be used to remove fluids, blood, or other secretions from the field of view to enhance visibility. These ports may be detachable to the outlet of the suction channel 306.

In another exemplary embodiment, a sensor may be located within the first section 307 of the device 300. In one embodiment, the sensor may be a pressure sensor. The pressure sensor may be used, for example, to detect if a threshold amount of pressure is being applied to the device to determine if the device should return to its non-bent straight position in order for the physician to remove the device for the patient's body cavity. In another embodiment, the sensor may be a temperature sensor, a saturation sensor, a force sensor, an airflow sensor, a pulse oximetry sensor, an oxygen sensor, and/or any other suitable sensor that may be desired to diagnosis, prevent, monitor, examine, and/or treat a disease or injury.

In one embodiment, the second section 317 of the device 300 may contain the connection 309 for example, the snap connection, necessary to join the first section 307 and the section 317 together. Within section 317 a PCB 308, an electrical connector 307, a battery 311, an USB Type-C connection 312 and a HDMI connection 313, and a power button 314.

The orientation of the device, as illustrated in FIG. 3 is only meant for exemplary purposes and may be arranged in any suitable manner.

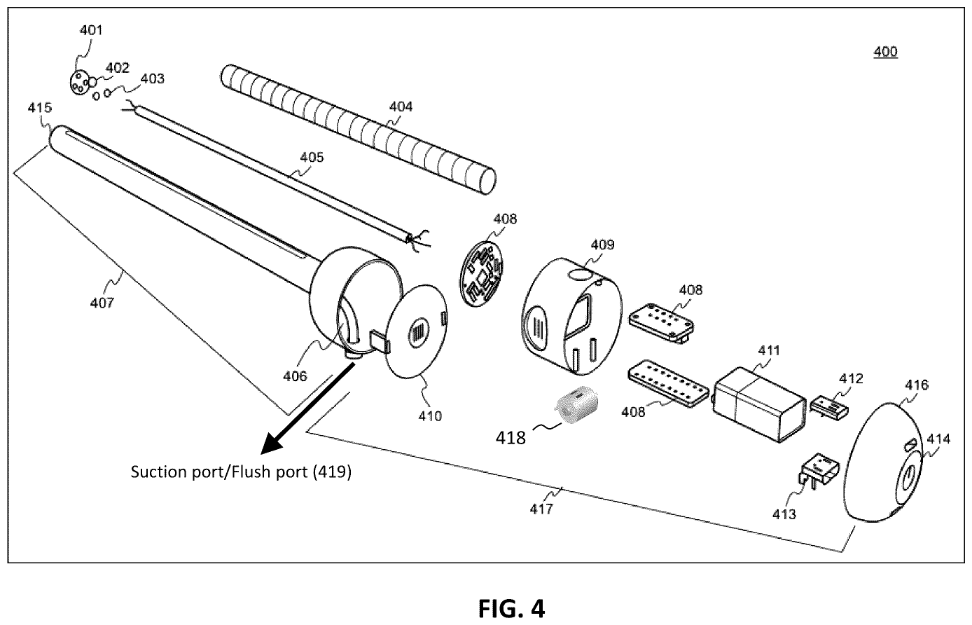

FIG. 4 is an exploded view of the endoscopic device which illustrates all of the same embodiments exemplified and described in FIG. 3 but also includes a motor 418. In one embodiment, motor 418 may be included, for example, to allow for the second section 407 of the device housing 400 to expand or retract to the optimal zone for which the tip 401 located at the distal end 415 of the device 400 should be located to collect data of the patient's anatomy. For example, as illustrated in the block diagram of FIG. 11, after analyzing the collected data, such as video received from the device, using machine learning, it could be determined that the length of the second section 407 of the device 400 needs to be expanded a 5 cm in order for the tip 401 located at the distal end 415 of the device 400 to be located in the optimal zone. Upon making this determination, a signal may be sent to device 400, in which device 400 will automatically expand the length of the second section 407 without any human intervention.

In one embodiment, the first section 407 of the device 400 may include a tip 401, a camera 402, a lighting source 403, electrical wires 405 connecting the camera 402 and light source 403 to the electrical connector 410, rings for bending 404, and a suction channel 406.

In one embodiment, the second section 417 of the device 400 may include an electrical connector 410, PCBs 408, a connection mechanism 409, a motor 418, a battery 411, USB Type-C connection 412, HDMI connection 413, and a power button 414.

The components located within the housing of the present invention may be arranged in any manner that is necessary to carry out the necessary functions of the device. For example, in one exemplary embodiment, the battery 411 may be located in the first section 407 of the device rather than how it is currently shown located in the figure in the second section 417.

In some embodiments, a suction port or flush port 419 may be attached to the suction channel 406. In some embodiments, the flush port may be used to deliver water, saline, or other liquid solutions to aid the visualization. Once inserted, the solution may clear any debris, mucus, or other obstructions to allow for a clear examination. The same may be performed using a suction port. In other embodiments, the suction port may also be used to remove fluids, blood, or other secretions from the field of view to enhance visibility. These ports may be detachable to the outlet of the suction channel 406.

The orientation of the device, as illustrated in FIG. 4 is only meant for exemplary purposes and may be arranged in any suitable manner.

FIG. 5 is a proximal perspective view of the endoscopic device 500 illustrating the second section 503 of the device 500 which, in one embodiment, rests outside of the patient's body cavity while in use. In this example, the first section 501 of the endoscopic device 500 may be inserted into a patient's body cavity in order to monitor the patient's anatomy. In one exemplary embodiment, the second section 503 of the device 500 may be reusable. The second section 503 of the device 500 may be attached to the first section 501 via for example, a snap lock 504. Other attachment means, such as screwing, twisting, clamping, are also contemplated. The first section 501 may detach from the second section 503 and may be reused for more than one procedure. In some embodiments, the reusable second section 503 may include all the electronic, control circuitry, communications circuitry for transmitting data to other displays and devices, extension and retraction modules for performing the extensions and retractions of the tubular portion, camera which can access the input from the tip of the second portion, AI modules for analyzing abnormalities, lighting modules to provide various forms of light while the endoscope is inserted into the patient, and gyroscope. Accordingly, the components in the reusable section may be retained for multiple uses since they may contain some of the key components that provide the operation of the endoscope and may be more expensive than the components in the second section. In another exemplary embodiment, the second section 503 of the housing may be disposable.

In one embodiment, as illustrated in the figure, the endoscopic device 500 may be in its straight, non-bent position. After the endoscopic device is inserted into the patient's body cavity the first section 501 may retract or expand in length. In another embodiment the first section 501 may also bend to different angles. The folds 502 of the device 500 represents the devices 500 capability of being able to expand or shorten in length upon a received signal to either auto-expand or auto-retract a determined length.

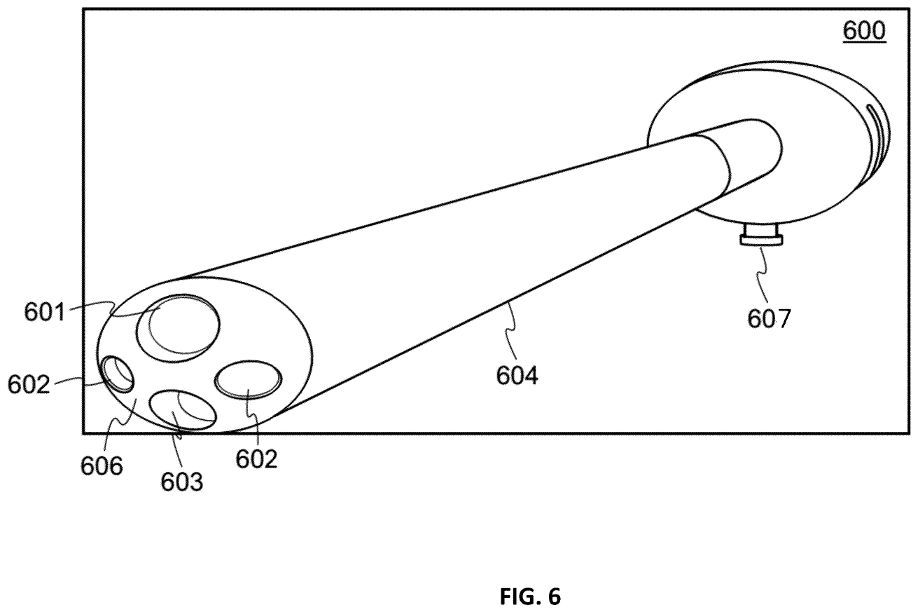

FIG. 6 is a distal perspective view of the endoscopic device illustrating the insertion portion of the device into the patient's body cavity. At the distal end of the device the first section 604, LEDs 602, and a camera 601 are shown. In this example, a domed tip 606 is shown. In other embodiments, any suitable tip for ease of insertion of the endoscopic device into the patient's body cavity may be used. In other embodiments, the endoscopic device 600 may contain more than one camera 601. Having more than one camera, for example, may allow for the device to have a wider range of view within the patient's anatomy. In another example, the device may contain one or more lighting sources 602. The lighting sources may be LEDS for example, or any other suitable light source to ensure visibility while the device is inserted in the patient's body cavity. The endoscopic device may also include a suction flushing port channel.

Although not shown, the endoscopic device may also include one or more sensors. In one embodiment, the sensors may be, for example, a pressure sensor. The pressure sensor may be used, for example, to detect if a threshold amount of pressure is being applied to the device to determine if the device should return to its non-bent straight position in order for the physician to remove the device for the patient's body cavity. In another embodiment, the sensor may be a temperature sensor, a force sensor, an airflow sensor, a pulse oximetry sensor, an oxygen sensor, and/or any other suitable sensor that may be desired to diagnosis, prevent, monitor, examine, and/or treat a disease or injury. In another exemplary embodiment, a sensor may be located within the first section 307 of the device 300.

FIG. 7 is an example of data from multiple patients, in accordance with some embodiments of the disclosure. As described earlier, the patient's depth or distance to the optimal zone from the point of insertion, as depicted in FIG. 11, and their diameter of the throat may differ from patient to patient. For example, as depicted in the table 700, for a 6 ft 5 in patient (Patient #1) who has a Haitian ethnicity, the distance from the point of insertion of the endoscopic device to the optimal zone may be 13 inches. The same Haitian patient may have an esophagus diameter of 24 mm.

In another example, for a 5 ft 3 in patient (Patient #2) who has a German ethnicity and weighs 150 lbs, the distance from the point of insertion of the endoscopic device to the optimal zone may be 11 inches. The same American patient may have an esophagus diameter of 17 mm. A second patient (Patient #3) who is also of American ethnicity and has the same height as Patient #2, but weight a lot more, i.e., 270 lbs, although the distance from the point of insertion of the endoscopic device to the optimal zone may be the same as Patient #2 due to them being the same height, i.e., 11 inches, the esophagus diameter may be larger due to their weight, e.g. 19 mm. Patient #4, of Sri-Lankan ethnicity, who may be about same height as Patient #2 and Patient #3 may have a different distance to optimal zone and diameter at optimal zone which may be due to genetics and their ethnicity.

As such, height, weight, gender, ethnicity, among other body measurements and backgrounds, may affect the patient's distance to optimal zone and diameter at optimal zone. The endoscopic device may automatically and without user intervention determine the patient distance to optimal zone and diameter at optimal zone, such as via providing visual input to an AI engine and obtaining related results. Based on the determined measurement of distance to optimal zone and diameter at optimal zone, the endoscopic device may automatically extend or retract to ensure that the tip of the endoscopic device is in the optimal zone and the orientation of the tip captures visuals in a clear manner in the esophagus.

The endoscopic device may also automatically control the speed of insertion, pauses during the process of insertion at various stages, and orientation at each paused state. For example, as the insertion is taking place and the tip of the endoscopic device has not yet reached the optimal zone, if any abnormality on the way is detected, the endoscopic device may automatically slow down and capture such images of the abnormality. The endoscopic device may also retract back to capture all angles of the abnormality. The amount of retraction may be automatically determined based on the size and nature of the abnormality. For example, if the abnormality stretches over a longer portion, the endoscopic device may detect the size of such abnormality and retract back enough to position the camera at the tip of the endoscopic device prior to the start of the abnormality to capture its image. The endoscopic device may then slowly proceed forward by automatically extending to capture several images from all angles. The endoscopic device may also capture a video. When such an abnormality, or anything else of concern, in the patient's esophagus is detected, the endoscopic device may automatically alert the caregiver, such as via a text, pop-up on their laptop etc. The endoscopic device may also invoke an AI engine executing an AI algorithm to determine whether the nature of the abnormality exceeds a threshold for transmitting an alert. The endoscopic device or an application used in conjunction with the endoscopic device, may not only provide an alert to the caregiver, but also provide details of the abnormality which may be determined from the AI engine.

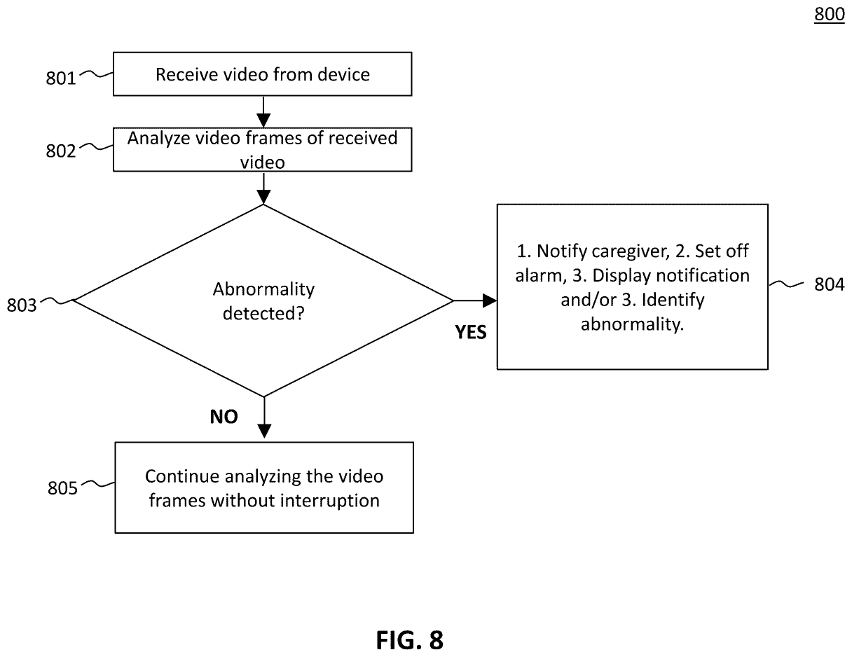

FIG. 8 is a block diagram illustrating, using machine learning, the steps followed if an abnormality is detected within the patient's anatomy. At step 801, the collected data is received from the endoscopic device 300. The collected data could be, for example, video taken from the camera 302 or pictures taken from camera 302. Once the collected data is received, the collected data, for example, video, is analyzed at step 802. At step 803, the analysis may include detecting abnormalities as shown at step 803. For example, using machine learning technology, the videos received from the camera 302 located in the device 300 may be processed by the processor 207 to determine the person's anatomy. In one embodiment, the processed video is analyzed to determine if any abnormalities within the patient's anatomy exist, for example, the machine learning technology may determine that the abnormality is eosinophilic esophagitis. In one embodiment, once the abnormality is detected, a notification may be displayed on the display screen indicating that an abnormality has been identified.

In another exemplary embodiment, the machine learning technology may determine that there is an abnormality with the patient's anatomy. For example, while analyzing the received video, the machine learning technology detects that the person has an abnormal septum and detects that the septum is deviated.

Turning to step 804, if the machine learning technology detects that an abnormality does exist, the caregiver may be notified that an abnormality was detected. An abnormality may mean, for example, any malformation, deformity, irregularity, that may be detected in the patient's anatomy. Further, in another embodiment, an alarm may be set off if an abnormality is detected. In yet another embodiment, a notification may be displayed, indicating that an abnormality was detected. In another exemplary embodiment, the abnormality may be identified. Identification may include outlining the abnormality on the display of the received video or picture received from the device 300. The identified abnormality may include the diagnosis, for example, on the display the abnormality may be identified as a deviated septum.

In yet another exemplary embodiment, the device may detect that due to an abnormality within the patient's anatomy, the placement of the device in a specific area in the patient's anatomy may cause an obstruction blocking the patient's airway which could result in, for example, suffocation. When the possibility of such an event is detected, the steps in block 804 may be followed. In addition to the steps in 804, the device may receive a signal instructing the device not to move beyond a certain point within the patient's anatomy.

Turning to step 805, if an abnormality is not detected, the received video from device 300 continues to be analyzed without any interruption.

FIG. 9 is a block diagram illustrating, using machine learning, placing the distal tip of the endoscopic device in the optimal zone within the patient's anatomy using auto-retraction and auto-expansion 900. At step 901, the collected data is received from the endoscopic device 300. The collected data could be, for example, video taken from the camera 302 or pictures taken from camera 302. Once the collected data is received, the collected data, for example, video data, is analyzed at step 902. At step 903, the analysis may include determining if the tip 302 located at the distal end 315 of the endoscopic device 305 is placed in the optimal zone within the patient's anatomy as shown at step 903. For example, using machine learning technology, the videos received from the camera 302 located in the device 300 may be processed by the processor 207 to determine the patient's anatomy. After determining the patient's anatomy, the optimal zone for the placement of the tip of the distal end of the endoscopic device may be determined. As used herein, the term “optimal zone” refers to the area within the patient's anatomy which is determined to be the best location for collecting data. For example, a basketball player who is 7 ft tall may have a different optimal zone of placement of the device than a gymnast who is 5 ft tall. The device 300 may analyze the received video to determine the patient's anatomy to determine if the tip 302 located at the distal end 315 of the endoscopic device 300 is placed within the optimal zone of the patient's anatomy.

At step 905, if it is determined that the tip 302 located at the distal end 315 of the endoscopic device 300 is not located within the optimal zone of the patient's anatomy, an adjustment of the tip may be determined. In some embodiments, for example, an adjustment may be defined as determining if the tip 302 located at the distal end 315 of the endoscopic device 300 should be expanded 2 cm to be located within the optimal zone. After determining the required adjustment, at step 906, a signal may be sent to the device 300 to either expand or retract to the optimal zone within the patient's anatomy.

At step 904, if the tip 302 located at the distal end 315 of the endoscopic device 300 is determined to be placed within the patient's optimal zone, the video continues to be analyzed.

FIG. 10 is a block diagram illustrating, using machine learning, where the distal tip of the endoscopic device should be placed within the patient's anatomy using tip adjustment 1000. At step 1001, the collected data is received from the endoscopic device 300. The collected data could be, for example, video taken from the camera 302 or pictures taken from camera 302. Once the collected data is received, the collected data, for example, video data, is analyzed at step 1002. At step 1003, the analysis may include determining if the tip 301 located at the distal end 315 of the endoscopic device 300 needs to be adjusted within the patient's anatomy as shown at step 1003. For example, using machine learning technology, the video data received from the camera 302 located in the device 300 may be processed by the processor 207 to determine the patient's anatomy. After determining the patient's anatomy, the placement of the tip 301 of the distal end 315 of the endoscopic device 300 may be determined if an adjustment is required. In some embodiments an adjustment of the tip located at the distal end 315 of the device 300 may include determining the optimal angle of the tip 301 in regard to the desired area within the patient's anatomy for which the device 300 is monitoring. For example, the device may be placed within the patient's determined optimal zone as shown in FIG. 11 but the angle of the tip of the device may require an adjustment. As used herein, the term “adjustment” refers to a small alteration or movement in the angle of the device made to achieve a desired result. For example, the device may receive a signal that the tip 301 of the device 300 should be adjusted 4 degrees to the right. At step 1006, once the signal is received the device will automatically adjust to the determined angle.

At step 1004, if it is determined that the tip of the device does not require an adjustment, the received video may continue to be analyzed.

FIG. 11 is an illustration depicting the optimal zone within a patient's anatomy of where the device should be placed 1100. As used herein, the term “optimal zone” refers to the area within the patient's anatomy in which it is determined to be the best location for collecting data. As depicted in the illustration, three different zones are shown. Zone 1101 depicts an exemplary first zone. In one embodiment, if the tip 1105 of the device 1104 were placed in this first zone 1101 the device would be placed too far of a distance from the optimal zone 1102. For example, if the tip 1105 of the device 1104 were placed in first zone 1101, the data that the physician is attempting to collect from the patient would not be the best data that the physician could collect due to the tip 1005 being too far away from the zone that is of most interest 1102. Zone 1102 depicts an exemplary second zone which is considered the optimal zone. The second zone 1002 is the best zone for the tip 1105 of the device 1104 to collect the necessary data from the patient. Zone 1103 depicts an exemplary third zone in which if the tip 1105 of the device 1004 were placed in this third zone the tip 1005 of the device 1104 would be placed too short of a distance from the optimal zone 1102. For example, if the tip 1105 of the device 1104 were placed in third zone 1103, the data that the physician is attempting to collect form the patient 1106 would not be the best data that the physician could collect which could lead to missed diagnosis. For example, if the physician has placed the device in the third zone 1105, the machine learning technology could analyze the imagery collected sent from the device 1104 and determine that the third zone 1103 is not the optimal zone 1102 in which the device should be placed. Therefore, the device may receive a signal indicating that the device should be expanded a certain, calculated distance that would place the tip 1105 of the device 1104 in the optimal zone 1102 to collect the best data of the patient's 1106 anatomy. It should be noted that the optimal zone may vary from person to person depending on many factors such as the patient's size, as also depicted in the table at FIG. 7. For example, a patient who is a full grown adult who is 6 ft tall would have a different optimal zone than a child who is only 4 ft tall. In one embodiment, using machine learning technology, the optimal zone can be calculated for each patient and the device 1104 may be able to automatically adjust the length and the angle of the device to be placed in the determined optimal zone for each patient without any human intervention.

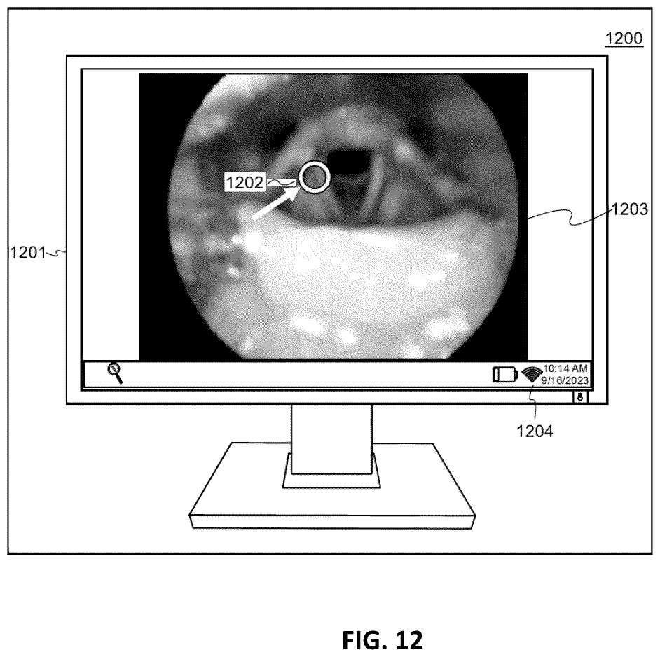

FIG. 12 is an illustration of an exemplary system 1200 of a user interface 1201 receiving the collected data 1203 from the endoscopic device wherein the machine learning technology has detected an identified abnormality 1202 within the patient's anatomy. For example, as depicted in the figure, the device 1104 of FIG. 11 has sent the collected data, such as video 1203, to the user interface 1201 in order for the machine learning technology to analyze the video 1203. As shown on the user interface screen 1201, an abnormality has been detected, exemplified by the circle and arrow 1202 outlining where the abnormality is located in the video 1203. In this example, the abnormality has been outlined by a circle and an arrow 1202. It should be noted that once an abnormality is identified, multiple actions may take place, for example, the caregiver may be notified, an alarm may be set off, the abnormality may provide a diagnosis, a notification may be provided on the display of the user interface, etc. In one embodiment, once an abnormality is identified, the device may also send additional data, such as additional pictures or videos, of that area within the patient's anatomy of the identified abnormality. The device, for example, may focus on that specific area, instead of analyzing other parts of the anatomy.

The present invention may be applied in any one or a subset of these approaches, or in a system employing other approaches for delivering data and commands. In one illustrative usage scenario, the endoscopic device 1104 of FIG. 11 may also be used for screening to check for diseases and health conditions before there are any signs or symptoms. For example, in one embodiment, the device may be used to screen for cancer before any signs or symptoms within the patient have appeared.

In another embodiment, the endoscopic device could be used in pre-anesthesia evaluations. For example, to avoid complications during the administration of anesthesia, the device could be used before the anesthesia is administered to determine if there is a complicated airway.

In another embodiment, the endoscopic device may be used for sleep apnea assessment. Typically, during the sleep apnea assessment, electrodes are placed on a patient's chest to determine if the patient stops breathing during their sleep but there is not any direct visualization into the airways. The device may be used to provide visualization into the airways during the study to understand, for example, if the airways are blocked and crowded.

In some embodiments, a method for using a wireless endoscope for inserting into an anatomical cavity is described. The method includes inserting, at least partially, a distal end of a first section of the wireless endoscope into the anatomical cavity. The first section is detachably connected to a second section of the wireless endoscope by a connection and the first section includes a tubular portion having an expanding and retracting mechanism within the tubular portion. The first section in some embodiments is disposable.

In this embodiment, the anatomical cavity is analyzed during insertion of the wireless endoscope. The analysis is performed as the wireless endoscope is being inserted.

For example, if the wireless endoscope is being inserted through the nose at a particular pace, then at each progression of the insertion, an analysis is performed to determine whether there is any abnormality. In some embodiments, the analysis may be performed at every centimeter as the wireless endoscope is inserted and in other embodiments the analysis may be performed if any obstruction is detected.

The analysis is performed automatically based on sensors in the wireless endoscope. It may also leverage artificial intelligence algorithms to determine whether an abnormality exists. For example, the artificial intelligence algorithm may compare each detection to medical books, opinions of physicians, and other medical data to determine whether the detection equates to an abnormality, such as a cancerous tissue.

Enroute to the complete insertion, the wireless endoscope may stop and retract when an abnormality is detected, and then wirelessly transmitting related data to an external device, such as a Physician's mobile phone, laptop, medical records, etc. The system may send a command to the expanding and retracting mechanism to stop or retract so it can take an image of the abnormality. The retraction may be to take images from all angles using the camera of the wireless endoscope. How much to expand and retract may be guided by the system, such as by using artificial indigence. Such guidance may be based on sensor data of the region (e.g., region where the expanding and retracting mechanism is stopped to take an image) in the anatomical cavity obtained by the sensors of the wireless endoscope.

The wireless endoscope may be automatically expanded using the expanding and retracting mechanism by a length until the distal end of the first section reaches a desired section of the anatomical cavity. The length of the expansion is based on the analyzed size of the anatomical cavity that may be analyzed by the system. How much to expand and to what length may be different for different patients based on their size of the anatomical cavity, which is automatically detected by the system leveraging artificial intelligence.

In some embodiments, the system may wirelessly transmit data relating to tests performed by the wireless endoscope while it is inserted into the anatomical cavity.

In some embodiments, the wireless endoscope automatically performs a suction using a suction channel of the wireless endoscope when detecting debris causing obstruction of view in the anatomical cavity.

In yet other embodiments, the wireless endoscope obtains a balance reading using a gyroscope located either in the first or second section of the wireless endoscope. The balance reading indicates whether the wireless endoscope is off balance or likely to slide of fall outside the anatomical cavity when not held or attached to something. As such, based on the balance reading, the wireless endoscope instructs a slidable weight to slide within the wireless endoscope to prevent sliding out of the wireless endoscope from the patient's anatomical cavity. For example, if a determination is made that the wireless endoscope may slide out, then the weight may slide to the end of the wireless endoscope, e.g., to towards the distal end of the inserted tip of the wireless endoscope that is inside the anatomical cavity, such that based on gravity of the weight, the wireless endoscope is weighted heavier inside the anatomical cavity than outside thereby preventing the wireless endoscope to slide out. The amount of sliding need may be dependent upon the balance reading and accordingly the system may instruct the weight to slide the requisite amount.

The above-mentioned assessments are only meant as exemplary situations in which the device may be used. The device may be used in any other assessments that may assist the clinician with screening, diagnosing, and treating patients. Furthermore, although references are made to patient and patient's anatomy, it may be applicable to any person or animal. Additionally, the components indicated to be in the first section may alternatively be placed in the second section of the housing and vice versa.

What is claimed is:

1. A wireless endoscope for inserting into an anatomical cavity, the wireless endoscope comprising:
a housing having a first section and a second section, wherein the first section is detachably connected to the second section by a connection, the first section comprises:
a tubular portion having a proximal end and a distal end, the distal end being configured to be at least partially inserted into the anatomical cavity;
an expanding and retracting mechanism within the tubular portion;
an electrical module functionally positioned at the distal end of the tubular portion comprising at least one camera; and
at least one suction channel located at the proximal end of the tubular portion.

2. The wireless endoscope of claim 1, wherein the expanding and retracting mechanism within the tubular portion automatically expands and retracts without user intervention to accommodate for a patient's anatomy.

3. The wireless endoscope of claim 2, wherein the expanding and retracting mechanism is instructed by an artificial intelligence which determines a length of expansion to reach an optimal zone in the anatomical cavity.

4. The wireless endoscope of claim 2, wherein the expanding and retracting mechanism automatically expands the tubular portion at a particular speed until the distal end of the tubular portion reaches an optimal zone in the anatomical cavity.

5. The wireless endoscope of claim 4, wherein the particular speed of expanding the tubular portion is varied by the expanding and retracting mechanism.

6. The wireless endoscope of claim 4, wherein the varying of the particular speed of expanding the tubular portion is based on encountering abnormalities along a passage of the anatomical cavity in which the tubular portion is inserted.

7. The wireless endoscope of claim 1, further comprising:
a release button located in the second section of the housing; and
in response to detecting a press of the release button, the expanding and retracting mechanism within the tubular portion retracting from a curved shape to a straight shape.

8. The wireless endoscope of claim 1, further comprising:
a gyroscope located either in the first or the second section of the housing; and
a slidable weight within the tubular portion shifted based on a reading from the gyroscope to prevent sliding out of the wireless endoscope from the anatomical cavity.

9. The wireless endoscope of claim 1, wherein the first section is disposable.

10. The wireless endoscope of claim 1, wherein the second section includes a camera and a lighting module.

11. The wireless endoscope of claim 1, further comprising control circuitry located in the housing, wherein the control circuitry is used for performing aspiration, swallowing, and cancer detection tests on a patient.

12. A method comprising for using a wireless endoscope for inserting into an anatomical cavity, comprising:
inserting, at least partially, a distal end of a first section of the wireless endoscope into the anatomical cavity, wherein the first section is detachably connected to a second section of the wireless endoscope by a connection and the first section includes a tubular portion having an expanding and retracting mechanism within the tubular portion;
analyzing, during insertion, a size of the anatomical cavity; and
automatically expanding the expanding and retracting mechanism by a length until the distal end of the first section reaches a desired section of the anatomical cavity, wherein the length of expansion is based on the analyzed size of the anatomical cavity.

13. The method of claim 12, further comprising, wirelessly transmitting data relating to tests performed by the wireless endoscope while it is inserted into the anatomical cavity.

14. The method of claim 12, wherein analyzing the size of the anatomical cavity is performed using sensors.

15. The method of claim 12, wherein analyzing the size of the anatomical cavity is performed using artificial intelligence.

16. The method of claim 12, further comprising:
during insertion, analyzing the anatomical cavity for an abnormality;
automatically stopping or retracting the expanding and retracting mechanism when the abnormality is detected;
capturing data relating to the abnormality; and
wirelessly transmitting data relating to the abnormality to an external device.

17. The method of claim 16, wherein the data related to the abnormality is detected by a camera of the wireless endoscope.

18. The method of claim 16, wherein the detection of the abnormality is performed by artificial intelligence using the captured data relating to the abnormality.

19. The method of claim 12, further comprising, automatically performing a suction using a suction channel of the wireless endoscope when detecting debris causing obstruction of view in the anatomical cavity.

20. The method of claim 12, further comprising:
obtaining a balance reading using a gyroscope located either in the first or the second section of the wireless endoscope; and
instructing, based on the balance reading, a slidable weight to slide within the wireless endoscope to prevent sliding out of the wireless endoscope from the anatomical cavity.
